(19) United States
(12) Patent Application Publication
(10) Pub. No.: US 2025/0118448 A1
(43) Pub. Date: Apr. 10, 2025

(54) Early Screening Model for Esophageal Cancer based on Oral Microbiota and Construction Method Therefor

(71) Applicant: SHANXI MEDICAL UNIVERSITY, Shenzhen (CN)

(72) Inventors: Matthew Yibo Cheng, Shenzhen (CN); Ning Ding, Shenzhen (CN); Heyang Cui, Shenzhen (CN); Ling Zhang, Shenzhen (CN)

(21) Appl. No.: 18/982,291

(22) Filed: Dec. 16, 2024

(30) Foreign Application Priority Data
Sep. 25, 2024 (CN) 2024113437636

(51) Int. Cl. G16H 50/30; C12N 15/10; G16B 35/20; G16H 50/70

(52) U.S. Cl. CPC G16H 50/30; C12N 15/1089; G16B 35/20; G16H 50/70

(57) ABSTRACT

The disclosure belongs to the technical field of biomedicine and analysis, and provides an early screening model for esophageal cancer based on oral microbiota and a construction method therefor. The model uses the following oral microbiota as markers: Prevotella, Granulicatella, Rothia, Prevotellamassilia, Blautia, Abiotrophia, Peptostreptococcus, Actinomyces, Burkholderia, Akkermansia, Fudania, Kineothrix, Bacillus, Duncaniella, and Eisenbergiella. The disclosure detects early asymptomatic esophageal cancer patients by identifying non-invasive biomarkers.

### TECHNICAL FIELD

The disclosure belongs to the technical field of biomedicine and analysis, and specifically relates to an early screening model for esophageal cancer based on oral microbiota and a construction method therefor.

### BACKGROUND

Esophageal cancer (EC) is a malignant tumor of the upper gastrointestinal tract that progresses from atypical hyperplasia to precancerous lesions, in situ carcinoma, and metastatic carcinoma. It ranks ninth among the most common cancers and sixth among causes of cancer death worldwide. Epidemic data reports that there were 604,100 new cases (6.3 per 100,000), 544,076 deaths (5.6 per 100,000) worldwide in 2020 and will increased to 957,000 new cases and 880,000 deaths in 2040. However, the main cause of such high incidence of EC has not been fully explored, resulting in a lack of primary etiologic prevention. Thus, the secondary prevention (early detection) has become the main approach for EC prevention. Early screening is one of the most effective methods for detecting cancer and saving lives. Research has shown that if detected early, 90% or more cancer patients can receive successful treatment. Unfortunately, the development of early screening strategies for esophageal cancer has been limited for decades, and the use of gastrointestinal endoscopy as the main method for early screening is still the only option currently available. In the process of upper gastrointestinal endoscopy, a thin and flexible tube (called an endoscope) with a small camera at the end is inserted from the patient's throat down into the esophagus. Being an invasive examination method to which the receivers have poor compliance, gastrointestinal endoscopy cannot be easily popularized, so esophageal cancer cannot be detected in a timely manner. In addition, due to the lack of specific symptoms in the early stage, most patients are diagnosed only in the middle and late stages, and rapidly progress to the late stage that treatment options are limited and cure is hopeless, resulting in high mortality rates. Together with the lack of intervention target for primary prevention, the status of clinical diagnosis of EC pulls out the urgent need for a non-invasive approach that can be easily used for early detection to control EC.

Oral microbiota, the second largest bacterial reservoirs in the human body, has been considered as a potential prediction biomarker for head and neck cancer, lung cancer, and colorectal cancer. Although previous studies suggest oral microbiota dysbiosis also present in EC patients, and poor oral hygiene is associated with an increased risk of EC, the predictive value of oral microbiota for esophageal cancer remains unclear due to the small sample size and a lack of periodontitis patients as another control besides a healthy control. Periodontitis is one of the risk factors of EC which also causes oral microbiota dysbiosis. Some pathogenic bacteria of periodontitis (such as Porphyromonas gingivalis, Fusobacterium nucleatum) not only exist in the cancer lesions but also can promote cancer progression by a variety of mechanisms, causing a pivotal issue which can affect the accuracy of the diagnosis/screening model. It is critical to determine whether the oral microbiome of EC patients is different from that of periodontitis patients. If the oral microbiome of those two groups were nearly the same, the existing biomarkers might wrongly detect periodontitis patients as EC patients. Therefore, it is necessary to recruit not only healthy people and EC patients, but also periodontitis patients to construct EC detection model by oral microbiome. This point is the key limitation of previous research and is the most important factor affecting the sensitivity and accuracy of EC detection model.

### SUMMARY

To address the current limitations of early screening methods for esophageal cancer patients, poor compliance of receivers, and difficulty in widespread popularization, as well as to solve the problem that using biomarkers to screen esophageal cancer cannot rule out the interference of periodontitis, the disclosure provides an early screening model for esophageal cancer based on oral microbiota.

The disclosure is implemented by the following technical solution: an early screening model for esophageal cancer based on oral microbiota is provided, using the following oral microbiota as markers: Prevotella, Granulicatella, Rothia, Prevotellamassilia, Blautia, Abiotrophia, Peptostreptococcus, Actinomyces, Burkholderia, Akkermansia, Fudania, Kineothrix, Bacillus, Duncaniella, and Eisenbergiella.

Optionally, relative abundance information of the oral microbiota is used as an

independent variable of the model, whether it is an esophageal cancer patient or not is used as a dependent variable of the model, a model is constructed using a Logistic regression model, and the formula of the model constructed by Logistic regression is: y=1/(1+e\hat{ }(2.89285631363524 +−0.130555223035498*Prevotella+−0.234624415130977*Granulicatella+−0.123421428328489*Rothia+0.0305284360696491*Prevotellamassilia+2.72774911990433*Blautia+−0.671714432121879*Abiotrophia+−0.643868902165489*Peptostreptococcus+1.81604325065481*Actinomyces+9.28516953416389*Burkholderia+−3.52739890519049*Akkermansia+9.46455532514789*Fudania+−7.93770666127306*Kineothrix+−127.099900821647*Bacillus+−93.3296419351167*Duncaniella+−37.9213542816937*Eisenbergiella)), where names of the microbiota in the formula refer to the relative abundance of the microbiota.

A method for constructing the early screening model for esophageal cancer based on oral microbiota, includes the following steps:

(1) collecting oral swab samples from healthy pariticipants, periodontitis patients and esophageal cancer patients;

(2) extracting DNAs from the oral swab samples and constructing libraries, performing library quality detection using a Qubit3.0 Fluorometer and an FEMTO Pulse system, and sequencing qualified libraries using a Pacbio Sequel platform;

(3) analyzing differences in compositions and structures of oral microbiota between by Bray-Curtis distance calculation method;

(4) constructing an early screening model (initial model): first, based on the whole cohort screening for species that distinguish the esophageal cancer patients using a random forest method (randomforest V4.6.12), where parameters are set as: randomForest obs˜, data=data, importance=T; Then, dividing the cohort into a discovery cohort and a validation cohort using a “Rand” function in EXCEL. Based on the discovery cohort constructing a model using a Logistic regression analysis, where a glm parameter of an R-3.6.3 function is set as: glm (obs˜., family=binomial ( ) data); determining classification performance of the initial model using area under curve (AUC), specificity and sensitivity in the receiver operating characteristic (ROC) curve;

(5) simplifying the model: examining an impact of excluding a single genus on the efficacy of the early screening model (initial model) for esophageal cancer by “leave one out” analysis; finally, trying to exclude four genera with low Mean Decrease Gini index and little impact on the evaluation performance for the model, constructing an early screening model as an ultimate simplified model using the Logistic algorithm, and validating the ultimate simplified model in the validation cohort. The AUC, specificity and sensitivity in the ROC curve are used as the performance evaluation indicators for the model.

The model has good screening effects on esophageal cancer. Specific parameters characterizing the screening effects are as follows: in the discovery cohort, the specificity is 0.839, the sensitivity is 0.911, the area under the ROC curve is 0.932 (95% CI is 0.900-0.965); and in the validation cohort, the specificity is 0.768, the sensitivity is 0.820, the AUC is 0.856(95% Cl is 0.806-0.906).

The method of the disclosure only requires collection of oral swab (saliva) samples from patients, and the subsequent sequencing, analysis. The diagnostic processes can be completed by professionals to achieve screening for esophageal cancer. The method of the disclosure has the advantages of non-invasiveness, convenience, and easy popularization. The receivers can complete oral sample collection at home by themselves, thereby achieving early detection and treatment of esophageal cancer.

The disclosure clarifies the differences in oral microbiota between esophageal cancer patients and healthy individuals as well as periodontitis patients, thus identify specific microbiota characteristics of esophageal cancer patients, and develop an efficient, specific, and sensitive early screening method for esophageal cancer. By recruiting 109 age-matched healthy volunteers, 138 periodontitis patients, and 224 esophageal cancer patients from Shanxi Province, a high-risk area for esophageal cancer in China, oral swab samples were collected, 16S-rDNA sequencing was performed, and the microbiota characteristics of the samples were analyzed. Subsequently, an early screening model based on oral microbiota was constructed with the relative abundances of indicator microbiota, and the diagnostic performance of the indicator microbiota was analyzed and evaluated using receiver operating characteristics (ROC). Therefore, the early screening model can successfully distinguish esophageal cancer patients from healthy individuals and periodontitis patients. The disclosure provides a non-invasive screening method for screening and early diagnosis of esophageal cancer, thereby providing patients with opportunities for early treatment. The disclosure detects early asymptomatic esophageal cancer patients by identifying non-invasive biomarkers, thereby improving poor prognosis and saving lives.

### BRIEF DESCRIPTION OF FIGURES

FIG. 1 is a quality control flowchart for sequencing data of microbiota from oral swabs.

FIG. 2 is a principal coordinate analysis plot (PCoA plot) of β-diversity differences in compositions and structures of oral microbiota between esophageal cancer patients, healthy individuals, and periodontitis patients.

FIG. 3 shows 20 genera were screened out by random forest analysis, that can be used to construct an early screening model for esophageal cancer.

FIG. 4 shows 19 genera, screened out by AUC analysis, can be used as indicator species for constructing the early screening model (initial model) for esophageal cancer.

FIG. 5 is a schematic diagram showing randomly dividing the research cohort into a discovery cohort and a validation cohort using a “Rand” function.

FIG. 6 shows the ROC curve of the early screening model (initial model) for esophageal cancer in the discovery cohort, with the AUC indicated as the area under the ROC curve.

FIG. 7 shows a schematic diagram illustrating examination of the impact of excluding a single genera on the EC screening model's diagnosis performance by “leave one out” analysis, where the genera marked in Bold represent excluded that have a significant impact on the performance of the screening model, and the genera marked with line represent those excluded that have an impact on the performance of the screening model. Ultimately, Solobacterium, Catonella, Filifactor, and Bergeyella were excluded, and the remaining 15 genera were used for model construction.

FIG. 8 shows the ROC curve of an early screening model (ultimate simplified model) for esophageal cancer constructed with 15 genera in the discovery cohort, with the AUC indicated as the area under the ROC curve.

FIG. 9 shows the ROC curve of the early screening model (ultimate simplified model) for esophageal cancer applied in the validation cohort, with the AUC indicated as the area under the ROC curve.

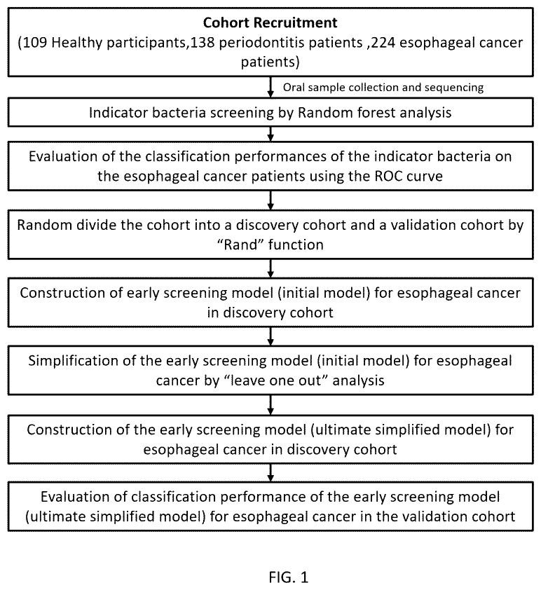

### DETAILED DESCRIPTION

To make the objectives, technical solution, and advantages of the examples of the disclosure clearer, the technical solution in the examples of the disclosure will be clearly described below. Obviously, the examples described are part of the examples of the disclosure, not all of them. Based on the examples of the disclosure, all other examples obtained by those of ordinary skill in the art without creative efforts shall fall within the protection scope of the disclosure.

Unless otherwise defined, all technical and scientific terms used herein have the same meanings as those commonly understood by those skilled in the art to which the disclosure belongs, and the references and materials cited herein will be incorporated by reference.

Equivalent technologies of the described specific implementations that can be understood through routine experiments, as recognized by those skilled in the art, will be included in the present application.

The experimental methods in the following examples, unless otherwise specified, are all routine methods. The instruments and equipment used in the following examples, unless otherwise specified, are all routine laboratory instruments and equipment. The experimental materials used in the following examples, unless otherwise specified, are purchased from routine biochemical reagent stores.

Reagents and consumables used in the examples are as follows:

TABLE 1
Brands and item numbers of reagents/materials used in examples

| Reagent/consumable | Brand | Item number |
|---|---|---|
| Hipure Universal DNA Kit | Guangzhou Magen | D3018 |
| | Biotechnology | |
| Sampling swab | AiDeTe | 50102032 |
| TransGen High-Fidelity PCR | TransGen Biotech | AS131-21 |
| SuperMix | | |
| AMPure PB Magnetic Beads | Beckman Coulter | 100-265-900 |

Instruments used in the examples are as follows:

TABLE 2
Brands and models of instruments used in examples

| Instrument | Brand | Model |
|---|---|---|
| PCR Amplifier | Eastwin Scientific | ETC811 |
| Qubit 3.0 | ThermoFisher Scientific | 3.0 |
| Centrifuge | Eppendorf | 5427R |
| Ultra-pure Water | Rephile Bioscience | Merck millipore ™-D |
| Purifier | | |
| Vortex mixer | Miulab | Miulab mix-28+ |
| Nanodrop Micro | ThermoFisher Scientific | Nanodrop2000 |
| Spectrophotometer | | |
| Agarose Gel | Beijing LIUYI | DYY-6C |
| Electrophoresis | | |
| Gel Imaging System | Shanghai Tanon | Tanon-2500 |

### I. Experimental Methods

1. Recruitment of research subjects: The cohort is of a case-control study design. From 2023 to 2024, 109 healthy individuals (H) were recruited at the First Affiliated Hospital of Shanxi Medical University, 138 periodontitis patients (P) were recruited at Shanxi Medical University School and Hospital of Stomatology, and 224 esophageal cancer patients (EC) were recruited at Shanxi Provincial Cancer Hospital. Oral swab samples were collected from the enrolled subjects, and information including gender, age, disease history, smoking and drinking history, tooth brushing frequency, and the like were collected by the staff.

The enrollment criteria for healthy individuals: 50 year old or older people who come to the hospital for physical examination, excluding: patients with obvious oral inflammation and periodontitis; patients who had bacterial or viral infections of the tonsils, salivary glands, or throat within one month prior to sampling; patients who received periodontal treatment within 3 months prior to sampling; patients who received general anesthesia surgery, radiotherapy, and chemotherapy in the past six months; patients with oral squamous cell carcinoma, head and neck squamous cell carcinoma, and colorectal cancer; and vulnerable groups such as pregnant women.

The enrollment criteria for periodontitis patients: patients (50 year old or older) diagnosed with periodontitis upon admission, excluding: patients with oral mucosal lesions; patients who had bacterial or viral infections of the tonsils, salivary glands, or throat within one month prior to sampling; patients who received periodontal treatment within three months prior to sampling; patients who received surgery, radiotherapy, and chemotherapy in the past six months; patients with oral squamous cell carcinoma, head and neck squamous cell carcinoma, and colorectal cancer; and vulnerable groups such as pregnant women.

The enrollment criteria for esophageal cancer patients: patients diagnosed with esophageal cancer upon admission, excluding: patients with obvious oral inflammation; patients who had bacterial or viral infections of the tonsils, salivary glands, or throat within one month prior to sampling; patients who received periodontal treatment within 3 months prior to sampling; patients who received general anesthesia surgery in the past six months; and vulnerable groups such as minors and pregnant women.

The information of all participants is shown in Table 3.

TABLE 3
Information of receivers

| | | | | Esophageal | |
|---|---|---|---|---|---|
| | | Healthy | Periodontitis | cancer | |
| | | individuals | patients | patients | p |
| | | (n = 109) | (n = 138) | (n = 224) | value |
| Age | Average | 65.57 ± 0.96 | 63.57 ± 0.88 | 65.14 ± 0.67 | ns |
| | value ± | | | | |
| | SEM | | | | |
| Gender | Male | 71 | 57 | 145 | <0.0001 |
| | Female | 38 | 81 | 79 | |
| Smoking | Yes | 42 | 34 | 130 | <0.0001 |
| history | No | 67 | 104 | 94 | |
| Drinking | Yes | 49 | 39 | 93 | 0.012 |
| history | No | 60 | 99 | 131 | |
| Tooth | Never | 2 | 0 | 11 | <0.0001 |
| brushing | Once | 23 | 2 | 142 | |
| frequency | a day | | | | |
| | ≥Twice | 84 | 136 | 62 | |
| | a day | | | | |

2. Collection method of oral swabs: Before sampling, the mouth was thoroughly rinsed with about 50 ml of water for about 10 s, and then the water was spit out. The swab was extended into the oral cavity, with the head of the swab fully touching the inside of the cheek and the upper and lower gum mucosae. The gum mucosae were rubbed up and down like brushing teeth, while the swab was rotated to ensure that the head of the swab fully contacted the oral mucosa. The operation was repeated for 1 min, and then the swab was put back into a sterile collection tube. The saliva samples were stored at an ultra-cold storage freezer with −80° C. until sequencing.

3. DNA extraction and library construction

A. DNA extraction (using Hipure Universal DNA kits for extraction, referring to the instructions for specific experimental methods). The following is a brief process:

1) The swab sample was transferred to a 2 ml centrifuge tube, and 500 μl buffer ATL (provided in the kit) and 20 μl protease K (provided in the kit) were immediately added and shaken in a warm bath at 56° C. for 1 h.

2) 500 μl buffer AL (provided in the kit) and 500 μl anhydrous ethanol were added and vortexed for 10 s.

3) Hipure DNA Mini Colum I (provided in the kit) was put into a 2 ml collection tube, and the mixture (750 μl or less) was transferred into the column and centrifuged at 10,000 g for 1 min.

4) The bottom waste liquid was discarded, the column was put back into the collection tube, the remaining mixture was transferred into the column and centrifuged at 10,000 g for 1 min, and the filtrate and the collection tube were discarded.

5) The bottom waste liquid was discarded, the column was put into a new collection tube, and 500 μl buffer GW1 (diluted with ethanol) was added to the column and centrifuged at 10,000g for 1 min.

6) The bottom waste liquid was discarded, the column was put back into the collection tube, 650 μl Buffer GW2 (diluted with ethanol) was added to the column and centrifuged at 10,000g for 1 min.

7) The bottom waste liquid was discarded, and the column was put back into the collection tube and centrifuged at 10,000 g for 3 min to dry the column.

8) The column was put into a 1.5 ml centrifuge tube, 50-100 μl Buffer AE (provided in the kit) preheated to 70° C. was added to the center of a membrane in the column, and the column was placed at room temperature for 3 min and centrifuged at 10,000 g for 1 min.

9) The DNA binding column was discarded and the DNA was used for subsequent experiments or frozen at −20° C.

B. PCR amplification: The extracted DNA was used as the template, and 16S V1-V9 primers (with barcode) included: 27F-AGRGTTYGATYMTGGCTCAG; 1492R-RGYTACCTTGTTACGACTT.

The amplification system included: 50 μl TransGen High-Fidelity PCR SupermMix containing 0.2 μM sense primers and anti-sense primers, and 5 ng DNA template. The amplification procedure is shown in Table 4.

TABLE 4
Amplification procedure

| | | | |
|---|---|---|---|
| 95° C. | 2 | min | |
| 95° C. | 30 | s | 35 cycles |
| 60° C. | 45 | s | |
| 72° C. | 90 | s | |
| 72° C. | 10 | min | |

C. Purification and recovery using AMPure® PB Beads: If the sample concentration or purity does not meet the standard, the amplification product could be purified and recovered using AMPure® PB Beads, and 1-2 μg amplification product was taken and transferred into a new 1.5 ml centrifuge tube.

The AMP PB Beads were taken out from 2-8° C. 30 min in advance and allowed to stand and equilibrate to room temperature. The AMP PB Beads were mixed thoroughly, and an equal volume of sample was pipetted and added to the screened amplification product sample. The sample was blown slowly and mixed evenly with a wide tip at room temperature for 15 times without lightly tapping the tube, and incubated at room temperature for 5 min. Following incubation, the sample was instantaneously centrifuged using a handheld centrifuge. The sample was placed on a magnetic rack and the supernatant was carefully removed after the solution is clear.

With the sample on the magnetic rack, 200 μl freshly prepared ethanol (80%) was added to rinse the magnetic beads, the sample was incubated at room temperature for 30 s, and the supernatant was removed. The previous step was repeated. The tube was removed and instantaneously centrifuged in the handheld centrifuge for 3 s. The sample was placed on the magnetic rack for about 10 s and the residual ethanol was carefully removed. With the sample on the magnetic rack, the magnetic beads were dried with an open lid at room temperature for about 5-10 min. The sample was removed from the magnetic rack, 45.5 μl Elution Buffer was added, and the sample was gently blown and mixed evenly with a wide tip, allowed to stand at room temperature for 15 min, and then placed on the magnetic rack until the solution is clear. 25 μl supernatant was carefully pipetted into a new nuclease-free EP tube.

D. Damage repair: The reaction system was as follows:

Amplification Product, 25.0 μl; DNA Damage Repair Mix v2, 2.0 μl; Total Volume, 27.0 μl. The reactants were incubated at 37° C. for 30 min, cooled down to 4° C. and stored to perform the next step.

E. End repair and addition of A: The reaction system was as follows: Amplification Product, 27.0 μl; End Prep Mix, 3.0 μl; Total Volume, 30.0 μl. The reactants were incubated at 20°° C. for 10 min, at 65° C. for 30 min, and stored at 4° C. to perform the next step.

F. Linker ligation: The reaction system was shown in Table 5. The reactants were incubated at 20° C. for 60 min and stored at 4° C. to perform to the next step.

TABLE 5
Reaction system

| | | |
|---|---|---|
| Amplification product | 30.0 | ul |
| Overhang Adapter v3 | 5.0 | ul |
| Ligation Mix | 30.0 | ul |
| Ligation Additive | 1.0 | ul |
| Ligation Enhancer | 1.0 | ul |
| Total Volume | 67.0 | ul |

G. Purification using AMPure® PB Beads: 3 μl Elution Buffer was added to the previous reaction system. The sample was mixed thoroughly, and 45 μl sample was pipetted and added to the ligation product. The sample was blown slowly and mixed evenly with a wide tip at room temperature for 15 times without lightly tapping the tube, and incubated at room temperature for 5 min. Following incubation, the sample was instantaneously centrifuged using a handheld centrifuge. The sample was placed on a magnetic rack and the supernatant was carefully removed after the solution is clear. With the sample on the magnetic rack, 200 μl freshly prepared ethanol (80%) was added to rinse the magnetic beads, the sample was incubated at room temperature for 30 s, and the supernatant was carefully removed. The previous step was repeated. The tube was removed and instantaneously centrifuged in the handheld centrifuge for 3 s.

Library quality detection was performed using a Qubit3.0 Fluorometer and an FEMTO Pulse system, and qualified libraries were sequenced using a Pacbio Sequel platform.

4. Analysis of differences in compositions and structures of oral microbiota between the esophageal cancer patients, healthy individuals and periodontitis patients: β diversity is an indicator that reflects the degree of differences in species composition and structural diversity between different ecosystems. In this implementation, statistical analysis of the β diversity was performed using a Bray-Curtis sample distance calculation method

$$\text{?}$$

$$\text{?} \text{indicates text missing or illegible when filed}$$

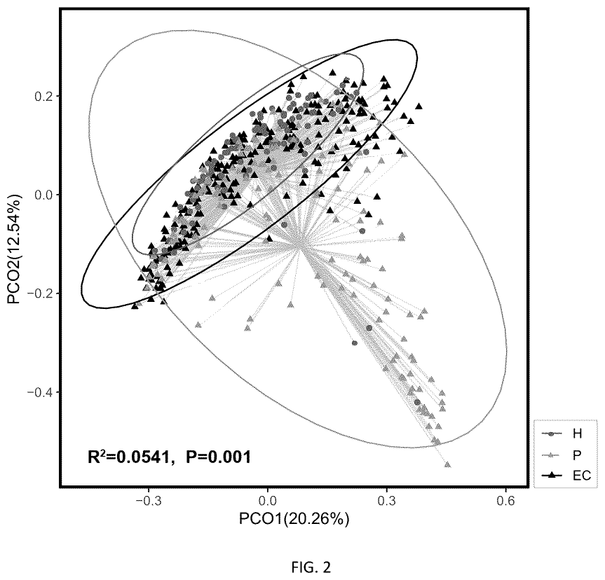

and the differences in microbiota composition and structure between different cohorts were presented using a PCoA plot (refer to FIG. 2). The Vegan software package in R language was mainly used for calculations and statistics.

5. Construction of an early screening model (initial model) for esophageal cancer: Firstly, based on the whole cohort, species that can distinguish the esophageal cancer patients were screened using a random forest method (randomforest (V4.6.12), parameters: randomForest (obs˜., data=data, importance=T)). Then, the full cohort was divided into a discovery cohort and a validation cohort using a “Rand” function in EXCEL. An early screening model (initial model) was constructed based on the discovery cohort using a Logistic regression algorithm (glm parameter of an R-3.6.3 function: glm (obs˜., family=binomial ( ) data)), and the corresponding model algorithm was generated. Classification performance of the model was determined through a receiver operating characteristic (ROC) curve.

6. Simplification of the initial model: An impact of excluding a single genus on e evaluation performance for the early screening model (initial model) for esophageal cancer was examined by “leave one out” analysis. Finally, four genera with low Mean Decrease Gini index and little impact on the performance evaluation indicators for the model were tried to be excluded, an early screening model was constructed as an ultimate simplified model using the Logistic algorithm, and the ultimate simplified model was validated in the validation cohort. Also, an area under curve (AUC), specificity and sensitivity in the ROC curve are used as the performance evaluation indicators for the model.

### II. Experimental Results

1. Existence of significant difference in the oral microbiota composition among esophageal cancer patients, healthy individuals, and periodontitis patients: By analysis of the β diversity of microbiota among three groups, it was found that the oral microbiota overall composition and structure of esophageal cancer patients was significantly different from the oral microbiota structures of healthy individuals and periodontitis patients. The difference was manifested in the PCoA PLot based on Bray-curtis distance, the sample distribution of the esophageal cancer patients was significantly different from those of the healthy individuals and periodontitis patients (P=0.001 according to statistical analysis) (see FIG. 2), indicating that the oral microbiota of the esophageal cancer patients differed in overall composition and structure from the non-esophageal cancer patients (including the healthy individuals and the periodontitis patients).

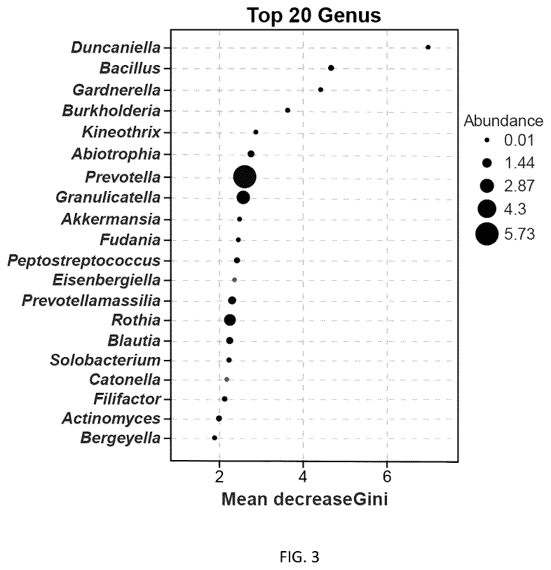

2. Screening of indicator species (genus level) that can be used for early screening and diagnosis of esophageal cancer using random forest analysis in the whole cohort: Firstly, the healthy individuals and the periodontitis patients were mixed as a non-esophageal cancer patient cohort. Further, optimal species at the genus level that can distinguish between the esophageal cancer patients and the non-esophageal cancer patients were screened using the random forest analysis, and ranked in a descending order based the Mean Decrease Gini index obtained from the random forest analysis. The top 20 genera (Prevotella, Gardnerella, Granulicatella, Rothia, Prevotellamassilia, Blautia, Abiotrophia, Peptostreptococcus, Actinomyces, Filifactor, Solobacterium, Bergeyella, Burkholderia, Akkermansia, Catonella, Fudania, Kineothrix, Bacillus, Duncaniella, and Eisenbergiella) ranked by the Mean Decrease Gini index were selected as the indicator microbiota for constructing the early screening model (FIG. 3).

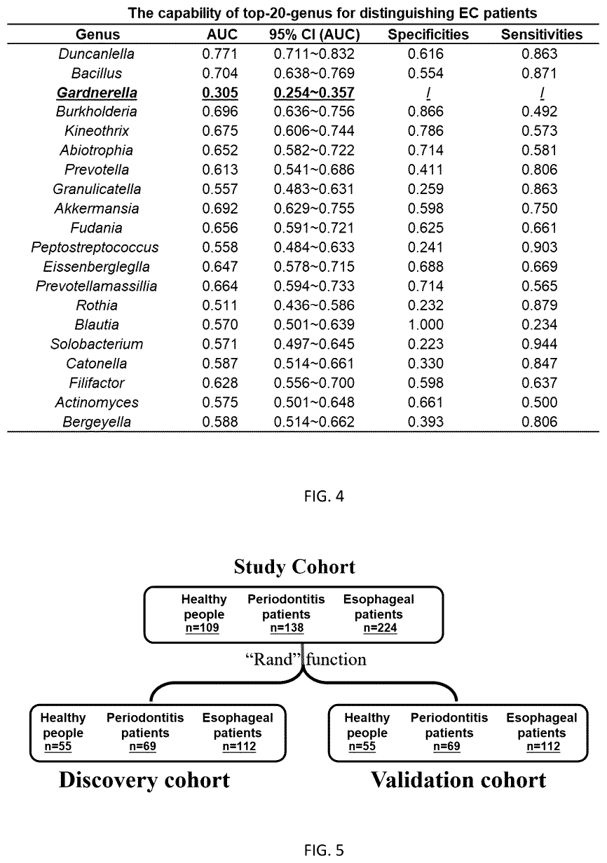

3. Evaluation of the diagnostic/screening/classification performances of the above indicator microbiota of 20 genera on the esophageal cancer patients using the ROC curve: The AUC is an indicator used to measure the effect of a genus in the diagnosis/screening/classification of esophageal cancer, generally ranging from 0.5 to 1.0. The closer the AUC is to 1.0, the better the effect of a genus in the diagnosis of esophageal cancer. In addition, specificity and sensitivity can respectively reflect the reliability and sensitivity of a species applied to the diagnosis/screening/classification of esophageal cancer. The higher the specificity, the lower the false positive rate, and the higher the sensitivity, the lower the false negative rate. From the results in FIG. 4, only Gardnerella has an AUC of 0.305, while the AUCs of other genera are greater than 0.5, indicating that Gardnerella does not have a good ability to distinguish esophageal cancer patients. Therefore, in the subsequent Logistic model construction analysis, Gardnerella was excluded and the remaining 19 genera (Prevotella, Granulicatella, Rothia, Prevotellamassilia, Blautia, Abiotrophia, Peptostreptococcus, Actinomyces, Filifactor, Solobacterium, Bergeyella, Burkholderia, Akkermansia, Catonella, Fudania, Kineothrix, Bacillus, Duncaniella, and Eisenbergiella) were used to construct the early screening model (initial model) for esophageal cancer.

4. Random divide the whole cohort into a discovery cohort and a validation cohort: The cohort of healthy individuals, periodontitis patients, and esophageal cancer patients was randomly divided into a discovery cohort and a validation cohort (see FIG. 5) using a “Rand” function in the EXCEL worksheet. The discovery cohort was used to construct an early screening model, and the validation cohort was used to revalidate the diagnostic performance of the constructed screening model.

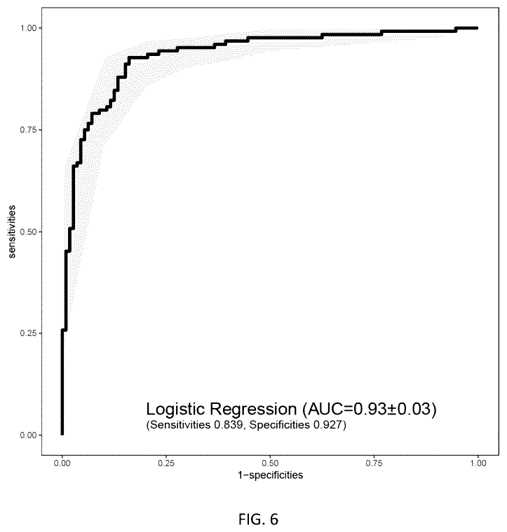

5. Construction of early screening model (initial model) for esophageal cancer: Firstly, in the discovery cohort, a model was constructed using a Logistic regression model for the 19 genera screened by the random forest model. The independent variable of the model was relative abundance information of the genera, and the dependent variable of the model was the disease grouping (whether it is an esophageal cancer patient or not). Following model construction through Logistic regression analysis, the predictive performance of the model was evaluated using the ROC curve. It was shown that the early screening model (initial model) constructed based on the 19 genera had high specificity (0.927), high sensitivity (0.839), and an AUC of 0.934 in the discovery cohort (FIG. 6), indicating that the early screening model constructed based on the 19 genera had excellent diagnostic/screening/classification abilities for esophageal cancer in the discovery cohort.

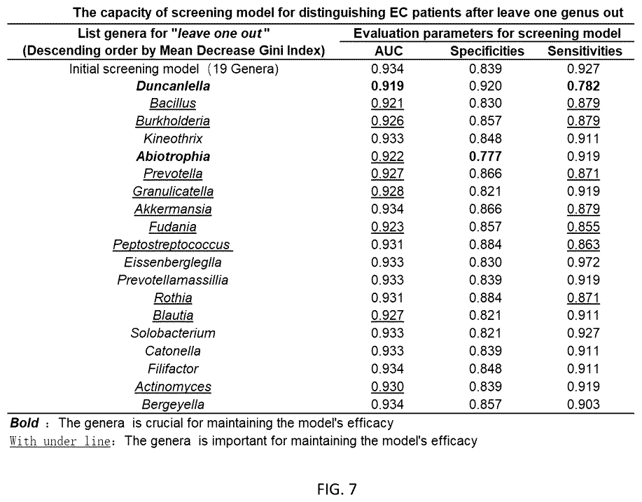

6. Simplification of the early screening model (initial model) for esophageal cancer: the impact of excluding a single genus on the esophageal cancer detection performance was examined by “leave one out”. The excluded genera that have a significant impact on the evaluation performance for the model were marked in Bold, and the excluded genera that have an impact on the evaluation performance for the model were with line (FIG. 7). Based on the Mean Decrease Gini index, finally, Solobacterium, Catonella, Filifactor, and Bergeyella were excluded, and the remaining 15 genera were used for constructing an early screening model (ultimate simplified) for esophageal cancer.

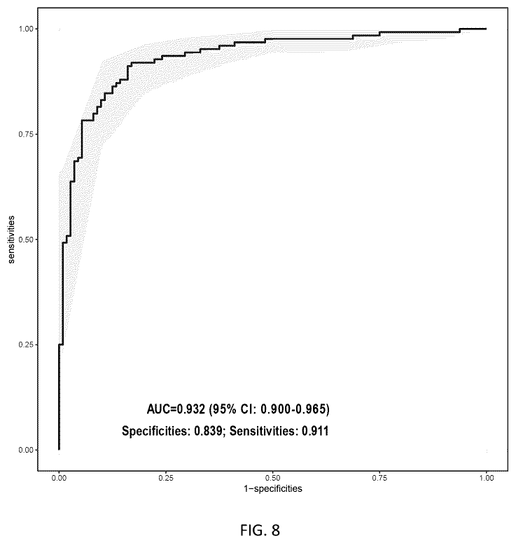

7. Construction of the early screening model (ultimate simplified model) for esophageal cancer: In the discovery cohort, a model was constructed through Logistic regression analysis for the screened 15 genera. The independent variable of the model was the relative abundance information of the genera, and the dependent variable of the model was the disease grouping (whether it is an esophageal cancer patient or not). Following model construction through Logistic regression analysis, the predictive performance of the model was evaluated using the ROC curve. It was found that the early screening model (ultimate simplified model) constructed based on the 15 genera had high specificity (0.839), high sensitivity (0.911), and an AUC of 0.932 in the discovery cohort (FIG. 8). Compared to the initial model before simplification, the AUCs are almost the same, the sensitivity has increased, and the specificity has decreased.

Overall, the early screening model (ultimate simplified model) for esophageal cancer still maintained good diagnostic/screening/classification abilities for esophageal cancer.

A formula of a model constructed by Logistic regression is: y=1/(1+e\hat{ }(2.89285631363524+−0.130555223035498*Prevotella+−0.234624415130977*Granulicatella+−0.123421428328489*Rothia+0.0305284360696491*Prevotellamassilia+2.72774911990433*Blautia+−0.671714432121879*Abiotrophia+−0.643868902165489*Peptostreptococcus+1.81604325065481*Actinomyces+9.28516953416389*Burkholderia+−3.52739890519049*Akkermansia+9.46455532514789*Fudania+−7.93770666127306*Kineothrix+−127.099900821647*Bacillus+−93.3296419351167*Duncaniella+−37.9213542816937*Eisenbergiella)), where names of the genera in the formula refer to the relative abundance of the genera.

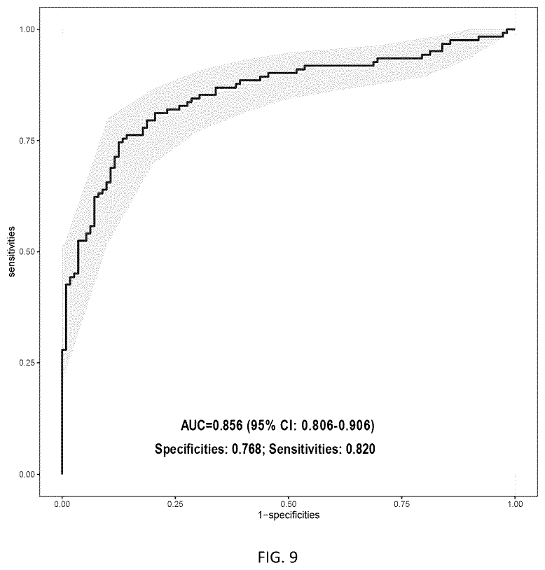

8. Evaluation of classification performance of the early screening model (ultimate simplified model) for esophageal cancer in the validation cohort: The constructed early screening model was further applied to the validation cohort and evaluated through the ROC curve (FIG. 9). It was found that the model had a specificity of 0.768, a sensitivity of 0.820, and an AUC of 0.856, indicating that the model still had good diagnostic/screening performances for esophageal cancer in an independent validation cohort.

Finally, it should be noted that the above examples are only used to describe rather than limit the technical solution of the disclosure. Although the disclosure has been described in detail with reference to the foregoing examples, those of ordinary skill in the art should understand that the technical solution described in the foregoing examples can still be modified, or some or all of the technical features can be replaced. And these modifications or replacements do not make the essence of the corresponding technical solution deviate from the scope of the technical solution of the examples of the disclosure.

What is claimed is:

1. An early screening model for esophageal cancer based on oral microbiota, using the following oral microbiota as markers: Prevotella, Granulicatella, Rothia, Prevotellamassilia, Blautia, Abiotrophia, Peptostreptococcus, Actinomyces, Burkholderia, Akkermansia, Fudania, Kineothrix, Bacillus, Duncaniella, and Eisenbergiella.

2. The early screening model for esophageal cancer based on oral microbiota according to claim 1, wherein relative abundance information of the oral microbiota is used as an independent variable of the model, whether it is an esophageal cancer patient or not is used as a dependent variable of the model, a model is constructed using a Logistic regression model, and a formula of a model constructed by Logistic regression is: y=1/(1+e\hat{ }(2.89285631363524+−0.130555223035498*Prevotella+−0.234624415130977*Granulicatella+−0.123421428328489*Rothia+0.0305284360696491*Prevotellamassilia+2.72774911990433*Blautia+−0.671714432121879*Abiotrophia+−0.643868902165489*Peptostreptococcus+1.81604325065481*Actinomyces+9.28516953416389*Burkholderia+−3.52739890519049*Akkermansia+9.46455532514789*Fudania+−7.93770666127306*Kineothrix+−127.099900821647*Bacillus+−93.3296419351167*Duncaniella+−37.9213542816937*Eisenbergiella)), where names of the microbiota in the formula refer to the relative abundance information of the microbiota.

3. A method for constructing the early screening model for esophageal cancer based on oral microbiota according to claim 1, comprising the following steps:
(1) collecting healthy oral swab samples, periodontitis oral swab samples, and esophageal cancer patient oral swab samples as analysis samples;
(2) extracting DNAs from the analysis samples and constructing libraries, performing library quality detection and sequencing qualified libraries;
(3) analyzing differences in composition and structure of oral microbiota between the qualified libraries by B-diversity using a Bray-Curtis sample distance calculation method;
(4) constructing an early screening model (initial model): dividing a full cohort into a discovery cohort and a validation cohort using a “Rand” function in EXCEL; first, based on the discovery cohort, screening for species that distinguish the esophageal cancer patients using a random forest method, constructing a model using a Logistic regression algorithm; determining classification performance of the initial model using an area under curve (AUC), specificity and sensitivity in a receiver operating characteristic (ROC) curve; and
(5) simplifying the model: examining an impact of excluding a single species on performance evaluation indicators for the early screening model (initial model) for esophageal cancer by “one by one exclusion”; finally, trying to exclude four species with low Mean Decrease Gini index and little impact on the performance evaluation indicators for the model, constructing an early screening model as an ultimate simplified model using the Logistic algorithm, and validating the ultimate simplified model in the validation cohort, where the AUC, specificity and sensitivity in the ROC curve are used as the performance evaluation indicators for the model.

4. The construction method according to claim 3, wherein early screening effects of the model on esophageal cancer are as follows: in the discovery cohort, the specificity is 0.839, the sensitivity is 0.911, the area under the ROC curve is 0.932 (95% Cl=0.900-0.965); and by evaluating the model with the ROC curve, in the validation cohort, the specificity is 0.768, the sensitivity is 0.820, the AUC is 0.856 (95% CI=0.806-0.906).
